(19) United States
(12) Patent Application Publication
(10) Pub. No.: US 2025/0082254 A1
(43) Pub. Date: Mar. 13, 2025

(54) SYSTEM AND METHOD FOR EVALUATING THE BRAIN'S RESPONSE TO SPOKEN LANGUAGE

(71) Applicants: CORNELL UNIVERSITY, Ithaca, NY (US); U.S. GOVERNMENT AS REPRESENTED BY THE DEPT. OF VETERANS AFFAIRS, Washington, DC (US)

(72) Inventors: Sudhin A. Shah, Ithaca, NY (US); Nicholas D. SCHIFF, Ithaca, NY (US); Nicholas Jeremy Hill, Albany, NY (US); James A. O'Sullivan, Dublin (IE); Edmund C. Lalor, Rochester, NY (US)

(21) Appl. No.: 18/718,132

(22) PCT Filed: Dec. 13, 2022
(86) PCT No.: PCT/US2022/081445

§ 371 Date: Jun. 10, 2024

Related U.S. Application Data
(60) Provisional application No. 63/289,076, filed on Dec. 13, 2021.

(51) Int. Cl. A61B 5/38; A61B 5/372; G16H 50/30

(52) U.S. Cl. CPC A61B 5/38; A61B 5/372; G16H 50/30

(57) ABSTRACT

The system and methods described herein diagnose the semantic processing capability of a subject by measuring the neural response of the subject to one or more naturalistic speech stimuli. The system measures the subject's temporal response function to the naturalistic speech by computing a statistical comparison between the subject's neural signal and a time series of semantic metric values corresponding to a transcript of the naturalistic speech stimulus. A diagnosis of the subject's semantic processing capability is then made based on an evaluation of the statistical comparison.

### CROSS-REFERENCE TO RELATED APPLICATIONS

This application claims priority to U.S. provisional application No. 63/289,076, filed Dec. 13, 2021, the content of which is hereby incorporated herein by reference in its entirety.

### BACKGROUND

Cognitive impairments are a devastating sequela following traumatic or non-traumatic brain injuries. Assaying residual and emerging cognitive function is especially challenging because it is dependent on motor function recovery. Currently, assessment of emerging and residual cognitive function is conducted via bedside behavioral assessments. These assessments are inextricably tied to co-emergence of motor function due to reliance on some type of motor output for responding, whether via eye, oral, or limb movements. This challenge is also borne out in settings where behavioral responses are limited by intercurrent illness, sedation, impaired motor/speech function, as is often encountered in critical care settings.

Accurately measuring cognitive function is of utmost importance for both protection of brain function and tracking of recovery. Inaccurate diagnoses have far-reaching impacts, especially in the acute post-injury stages where the early detection of preserved cognitive function, through its positive effect of prognosis, has a profound influence on time-sensitive end-of-life decisions and the allocation of appropriate resources including access to rehabilitation. Sensitive assessments are also critical to the identification of cognitive impairments that, if unidentified, create barriers to rehabilitation efforts. Chronically, the accurate diagnosis of preserved cognitive function would enable identification of persons, living in isolation, for whom alternative means of communication (e.g. brain computer interfaces) can and should be developed.

Thus, there is an unmet need to uncouple cognitive assessments from motor function and directly measure high-level human information processing and such systems and methods would make it more feasible to assess cognitive processing especially in pediatric populations.

### SUMMARY OF DISCLOSURE

According to one aspect, the disclosure relates to a method comprising recording neural data of a subject while the subject is presented with a natural speech stimulus; obtaining the transcript of the natural speech stimulus, with data indicating onset time of words included in transcript; calculating using a processor a semantic metric for words used in the transcript by relating the meaning of the words to its preceding context; creating using a processor a time series of impulses based on the onset times of the words in the transcript and the semantic metrics calculated for the words; calculating by the processor a stimulus-response mapping function by regressing the recorded neural data onto the time series; and determining by the processor the semantic processing capability of the subject based on the calculated mapping function.

In one aspect, the semantic metric for the words used in the transcript is calculated by relating the meaning of the words used in the transcript.

In one aspect, the stimulus-response mapping function is calculated using regularized linear regression.

In some aspect, the semantic processing capability of the subject is determined based on the identification or absence of a peak in the stimulus-response mapping function at a time of about 300 to about 400 ms.

In some aspects, the semantic processing capability of the subject is determined based on the identification or absence of a peak in the stimulus-response mapping function at a time lesser than 300 ms or greater than 400 ms.

In some aspects, determining the semantic processing capability of the subject based on the calculated mapping function includes determining the mapping function having a statistically significant correlation between the neural data and the time series than a random mapping function.

In another aspect, the semantic metric for a word in the transcript is based on the probability that a given word will follow preceding words in the transcript.

In one aspect, the semantic metric for a word in the transcript is based on a difference between a vector indicative of the semantic meaning of the word relative to one or more semantic vectors or combinations thereof corresponding to preceding words in the transcript.

In another aspect, the semantic metric for a word in the transcript is based on the probability that a given word will follow other words in the transcript.

In one aspect, the semantic metric for a word in the transcript is based on a difference between a vector indicative of the semantic meaning of the word relative to one or more semantic vectors or combinations thereof corresponding to other words in the transcript.

In one aspect, the method further comprises after determining the semantic processing capability of the subject, administering a medical treatment to the subject; after administering the treatment, receiving, by the processor, a measurement of a second neural response of a subject to one or more second naturalistic speech stimuli; receiving, by the processor, information related to the one or more second naturalistic speech stimuli; determining, by the processor, a second statistical relationship between semantic contribution of words in the second naturalistic speech stimuli to the second neural response; identifying, by the processor, a second semantic processing capability of the subject based on the determined second statistical relationship; and comparing, by the processor, the determined first semantic processing capability to the determined second semantic processing capability function; determining, by the processor, an efficacy of the medical treatment based on the comparison; and, outputting, by the processor, the determined efficacy of the medical treatment.

In one aspect, the disclosure relates to a system comprising a processor for generating a transcript of natural speech stimulus presented to a subject and for annotating the transcript with an onset time of words in the transcript; a neural sensor for recording a neural response of the subject to the natural speech stimulus; one or more processors implementing a processing unit configured to determine an indication of the semantic processing capability of a subject to the one or more naturalistic sensory stimuli by: receiving a measurement of a neural response in the subject exposed to the one or more naturalistic speech stimuli output by the neural sensor; determining a statistical relationship between the semantic contribution of words in the transcript to the naturalistic sensory stimuli and the measurement of the neural response of the subject; determining an indication of the semantic processing capability of the subject based on the statistical relationship; and, an output module for outputting the determined semantic processing capability.

### BRIEF DESCRIPTION OF THE DRAWINGS

The foregoing will be apparent from the following more particular description of example implementations of the invention, as illustrated in the accompanying drawings. The drawings are not necessarily to scale, emphasis instead being placed upon illustrating implementations of the present invention.

FIG. 1 is a diagram of an environment for assessment of an event-related brain response in a subject is provided according to an example implementation.

FIG. 2 shows a flow diagram of a method for assessing semantic temporal response functions (TRF) in subjects according to an example implementation.

FIGS. 3A-3D illustrate plots of temporal response functions of two subjects in response to discrete speech stimuli and continuous speech stimuli.

FIG. 4 shows a flow diagram of an example method for using signals reflective of language processing as objective markers of cognitive processing that can be used to determine the efficacy of a medical treatment.

FIG. 5 illustrates a block diagram of an example computing system.

For purposes of clarity, not every component may be labeled in every figure. The drawings are not intended to be drawn to scale. Like reference numbers and designations in the various figures indicate like elements.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The present disclosure relates to methods to characterize recovery of cognitive function. More particularly, the systems and methods of the disclosure relate to assessing semantic processing functionality of a subject based on determining a relationship between neural response (as measured by EEG, MEG, ECOG or the like) and the syntactic and semantic characteristics of words in spoken language. This could include isolating brain responses to incongruent words, identifying frequency-based brain responses to isochronously presented speech tokens, or deriving a semantic temporal response function based on computational linguistics measures of natural speech, as described further below.

Specifically, this disclosure relates, at least in part to methods of assessing semantic processing that is uncoupled from motor function in a subject, and is particularly, though not exclusively, well-suited for use in a pediatric population. More particularly, the systems and methods of the disclosure relate to assessing semantic processing functionality of a patient based on a semantic temporal response function (TRF) obtained by determining a relationship between neural response (as measured by EEG, MEG, ECOG or the like) and the syntactic and semantic roles of words in natural language, as described further below.

Systems and methods according to the present disclosure provide an electroencephalography (EEG), magnetencephalography (MEG) or electrocortiography (ECoG) based diagnostic for the assessment of language processing in the brain function of subjects presented with natural-language stimuli. In some implementations, the stimuli may be an auditory stream of speech. The speech may be spoken live, or played from a recording. In various implementations, the speech may be extemporaneous, scripted to replicate natural speech, the audible reading of the text of a book, story or other text, or other natural speech content. In other implementations, the stimuli could be a musical stimulus. The music may be played live or from a recording

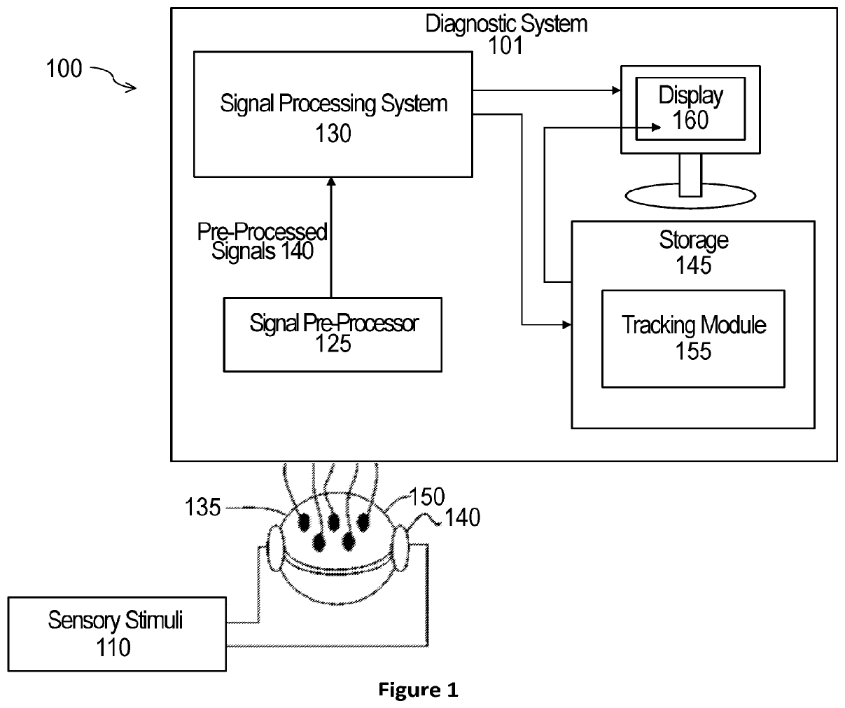

FIG. 1 is a diagram of an example environment 100 for diagnosing a patient based on an auditory semantic processing analysis. FIG. 1 shows a subject 135 presented with a natural speech stimulus 110. In some implementations, the natural speech stimulus 110 may be extemporaneous speech. In some implementations, the natural speech stimulus 110 may be text read out loud by a caregiver, where the text may include a story of interest to the patient. In some implementations, the natural speech stimulus 110 may be by a caregiver naturally speaking to the patient. In some implementations, the speech stimuli may be a radio program, podcast, audio book, the audio of a television program, a movie, or other item of media. In FIG. 1, the subject 135 is presented with a stream of natural speech stimulus 110 via a headset 140. In some implementations, the natural speech stimuli is ambiently presented speech, for example, spoken by a person in the vicinity of the patient or output via a loud speaker stimulus 110.

The environment 100 includes a wearable sensing system 150 such as a wearable EEG sensing system. Such wearable sensing system may include, but not limited to dry EEG systems and wet EEG systems. The sensing system 150 is positioned on the scalp of the subject 135 and acquires the brain signals of the subject 135 in response to the natural speech stimulus 110. In some implementations, the sensing system 150 is an EEG, MEG or ECOG based system. In some implementations, the sensing system 150 may have 24 or 7 EEG sensors positioned along the International 10/20 system. In other implementations, other numbers of EEG sensors and placement locations can be used. The brain signals acquired by the sensing system 150 are amplified, filtered, and digitized via an analog-to-digital converter. The environment 100 includes a diagnostic system 101. The diagnostic system includes a signal pre-processor 125, and a signal processing system 130. The signal pre-processor 125 automatically removes artifacts from the brain signals acquired by the sensing system 150. In some implementations, the signal pre-processor 125 may utilize an independent component analysis (ICA) for artifact removal. In some implementations, artifacts may be removed by visual inspection. In other implementations, values that exceed a certain amplitude may be considered artifacts. The signal pre-processor 125 samples the acquired brain signals at a sampling rate. In some implementations, the sampling rate is equal to or above 250 Hz. In other implementations, the sampling rate is below 250 Hz. Although not shown in FIG. 1, in some implementations, the environment 100 also includes an amplifier, a digitizer, a recording device, a speech transcription system, an application programming interface, sensors, and presentation computers or laptops that time-lock the presentation of the speech with the acquisition of the neural data. In some implementations, presentation software such as a neurobehavioral system is used to time-lock the presentation of speech stimuli. Preferably, the neurobehavioral system is able to time-lock the neural signal to speech to within the order of several milliseconds.

The diagnostic system 101 also includes the signal processing system 130. The signal pre-processor 125 generates pre-processed brain signals 140. The pre-processed brain signals 140 and the stimulus 110 are input into the signal processing system 130. The signal processing system 130 processes the pre-processed brain signals 140 in order to compute the event-related brain response of the subject 135 to the stimulus 110. The signal processing system 130 computes the event-related brain response and can extract signal features of the event-related brain response. The extracted signal features can include the latencies, amplitudes, polarities, and spatial distribution of the event-related brain response. As used herein, the spatial distribution of the event-related brain response refers to the manner in which the event-related brain response varies from EEG channel to EEG channel placed on different locations on the subject's scalp.

The diagnostic system 101 also includes a memory storage unit 145, a tracking module 155, and a display 160. In some implementations, the signal processing system 130 may store data and results in the memory storage unit 145 for offline analysis. In some implementations, the stored data in the memory storage unit 145 may be tracked over time through the tracking module 155. The tracking module 155 may track multiple measurements of the sensory evoked response based on different naturalistic sensory stimuli or different trials of the same naturalistic sensory stimuli over time. In some implementations, the signal processing system 130 may dynamically compute and present the real-time results on the display 160. In some implementations, the results may include the extracted signal features, the classification of the patient condition, and classification of the semantic processing capability of the subject. In some implementations, the results may also be actively displayed during a patient screening, in an emergency room setting following severe brain injury, or as a measure to track the patient's recovery and response to existing and novel treatments. For tracking purposes, any of the features of the event-related brain response, including latencies, amplitudes, polarities, and spatial distribution, may be stored in the storage 145 over time and compared by the signal processing system 130 or tracked by the tracking module 155. The results of the comparison may be displayed on the display 160, for example as a trend line, a graph, or a textual or graphical representation of the comparison results.

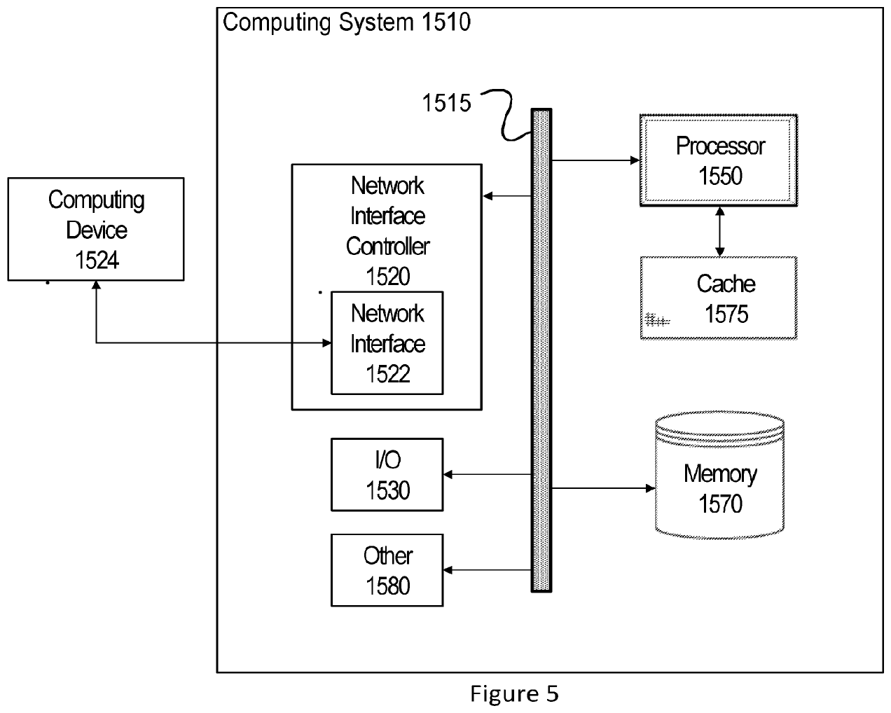

The analysis of the subject's language processing capability may be provided by a computer and outputted by the computer for example via the display 160, a printer, or over a computer network. Details describing a suitable architecture for such a computer system are described further in FIG. 5 below.

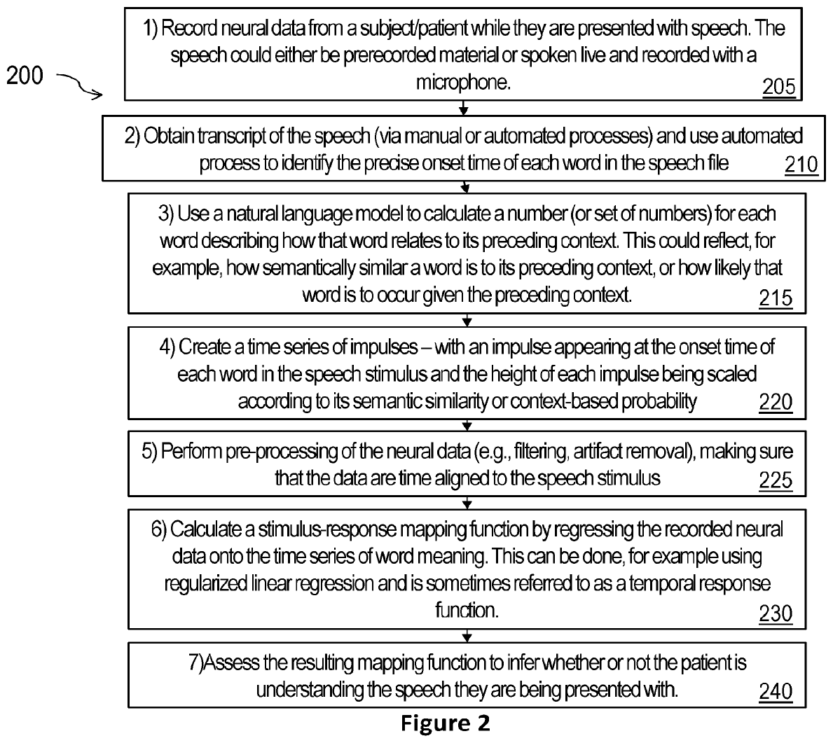

FIG. 2 shows a flow diagram of an example method 200 for providing a natural speech temporal response function evaluation for the assessment of brain function according to an example implementation. Referring back to FIG. 1, the method 200 includes recording neural data of a subject 135 while the subject is presented with a sensory stimuli such as, but not limited to speech. The speech could either be prerecorded or spoken live and recorded with a microphone stimulus 110 (step 205). The method 200 also includes obtaining the transcript of the speech from the subject 135, which can be done either manually or using an automated process. The precise onset time of each word obtained in the speech file is then identified using an automated process (step 210). The method 200 includes using a natural language model to calculate either a number or a set of numbers (e.g, a high-dimensional vector) for each word described in the speech file and understand how each word relates to its preceding word in the context. This could reflect, for example, how semantically similar a word is to its preceding context, or how likely that word is to occur given the preceding context (step 215). In some cases, the method 200 includes using a natural language model to calculate either a number or a set of numbers for each word described in the speech file and understand how each word semantically relates to other words (e.g., words other than its preceding word) in the speech. The method 200 also includes creating a time series of impulses. An impulse appears at the onset time of each word in the speech stimulus and the height of each impulse is scaled according to its semantic similarity or context-based probability (step 220). The method 200 includes performing pre-processing of the neural data, such as, but not limited to filtering, artifact removal in order to make sure that the data is time aligned to the speech stimulus (step 225). The method further includes calculating a stimulus-response mapping function by regressing the recorded neural data onto the time series of word meaning. This can be done, for example, using regularized linear regression and is sometimes referred to as temporal response function (TRF) (step 230). The method 200 includes assessing the resulting mapping function to infer whether or not the subject 135 is understanding (i.e., is semantically processing) the speech they are being presented with.

Referring back to FIG. 2 in more detail, the method 200 begins with step 205. Step 205 includes recording neural data from a subject 135 while the subject is being presented with speech. This speech could be any prerecorded material, such as, but not limited to audiobooks, podcasts. This speech could also be from a live speaker, in which case, it should be recorded concurrently with the neural data. In both cases, it is important that the neural recordings are “tagged” with one or more markers indicating the timing of the speech stimulus presentation relative to those neural recordings. For example, this could take the form of a simple tag at the beginning of the presentation of the speech stimulus.

An advantage of this approach described in step 205 is the ability to use engaging, subject-appropriate speech material that is likely to be of interest to the subject 135, enhancing the chances of identifying neural signatures of conscious understanding. This could include recording a family member or a loved one reading or talking extemporaneously. This could also involve using pre-recorded audiobooks or radio broadcasts that might be of interest to the subject.

The neural data in step 205 could likely be electroencephalography (EEG) data, although other methods for recording neural data could possibly be used. In the case of EEG data, one might record from many EEG channels, or one could may record from as few as two, with one of these ideally being placed over midline parietal scalp, and a second being placed at a reasonable distance from the first e.g., on the mastoid process behind an ear, over frontal midline scalp, on the nasion.

The method of 200 includes obtaining the transcript of the speech as disclosed in step 210. In order to analyze electrical brain responses to a specific stimulus, it is important to know when those stimuli occurred to within a few milliseconds. Because we are interested in responses to words (and their meaning), the method involves identifying (within a few milliseconds) the onset time of each word in the speech stimulus. This can be done using appropriate existing software. One first obtains a transcript of the audio speech—which can be done manually or using natural speech recognition software—and an audio file corresponding to the speech itself. The audio speech file and its transcript are provided to the diagnostic system 101. The output can be manually checked for accuracy.

The method of 200 further includes step 215 which involves the calculation of how each word in the speech file relates to its preceding context. This can be achieved, for example, by using a natural language model. The field of natural language processing by deep neural networks is a rapidly advancing one. These systems are based on training deep neural networks (DNNs) to recognize patterns in huge bodies of text. One common feature of these approaches is the modeling of word meaning as a vector of numbers. One approach, for example, determines these vectors based on how often different words co-occur in the training text. In this way, the vector “embeds” the meaning of the word, and the meaning of two words can be compared based on how similar their vectors are. The “amount” of meaning a word injects to a narrative can then, for example, be quantified by calculating how dissimilar its vector is to the vectors of the preceding words in the narrative (Broderick et al., 2018). Thus, the current word can be tagged with a single number representing its semantic dissimilarity to its preceding context.

Other deep neural network-based natural language processing systems are specifically trained to predict an upcoming word based on previous context. Once again, these networks typically embed word meaning in vectors of numbers. But they also provide a direct measure of the probability of a particular word occurring given some preceding words. In this way, the current word can be tagged with a single number representing its probability of occurrence given its preceding context. This next-word probability measure does not necessarily correlate with the semantic dissimilarity measure discussed above. They may provide complementary information. Indeed, a single word can be tagged with both measures (semantic similarity, probability).

The method of 200 includes step 220, creating a time series of impulses. A stimulus time series is created that initially consists of zeroes at the same sampling rate as the recorded neural data—e.g., 500 Hz. At the onset time of every word in the stimulus an impulse is added to the time series that is scaled according to the semantic relatedness of that word to its preceding context (as described in step 215 above). Thus, one can obtain a time series of impulses of varying magnitudes at the same sampling rate as the neural data with the impulses denoting the timing and context-based semantic value of every word.

The method of 200 further includes step 225, performing pre-processing of the neural data obtained from step 205. Preprocessing the neural recordings can be done by standard methods, including filtering the data into relevant frequency ranges and removing noise artifacts from the data. This step also ensures that the neural recordings from step 205 are precisely time aligned to the stimulus time series created from step 220. It is valuable to know which neural data points correspond to the presentation of which words in the stimulus.

The method of 200 further includes step 230, calculating a stimulus-response mapping function. It is assumed that the recorded neural data r(t) can be mathematically related to the stimulus time series s(t). Under this assumption, a mapping function can be derived that relates s(t) to r(t) according to some constraints. For example, one can assume that s(t) maps to r(t) according to a linear time-invariant mapping. Such a mapping can be estimated using (regularized) linear regression, in which case it is sometimes referred to as a temporal response function. Importantly, the function seeks to index how the neural response data reflect the stimulus at different relative time lags between the stimulus and response. As such, the function should be close to zero when considering time lags where the neural data occur before the stimulus. Non-zero values of the function at time lags where the neural data follow the stimulus by around 300 to 700 ms are of particular interest for studying language understanding. The mapping function can be derived separately for each neural recording channel. In EEG data, the function typically consists of a series of voltage fluctuations that can be assessed in terms of their amplitude and latency. Depending on the number of neural channels recorded, it can also be assessed in terms of its distribution on the scalp or in the brain. In some aspects, the stimulus-response mapping function includes a statistically significant correlation between the neural data r(t) and the time series s(t) than a random stimulus-response mapping function.

The method of 200 concludes with step 240, assessing the resulting mapping function from step 230. The features of the mapping function derived in the step 230 can be assessed to infer the likelihood that the patient was attending to and understanding the speech they were presented with. Evidence of understanding can be gleaned, in particular, from the polarity and amplitude of the mapping function over midline parietal scalp at time lags of around 300-700 ms between the stimulus and the neural data, as discussed above. Large negative values of the mapping function over midline parietal scalp (relative to other parts of the scalp) present strong evidence of language understanding. The latency (timing) of the maximum absolute value of this mapping function can also be used to infer attentiveness to the stimulus and efficient language processing.

Furthermore, once a mapping function is derived from step 240, the function can be used to “predict” neural responses in a patient to a new speech stimulus. Accurate predictions of neural responses are strong evidence of language understanding. Importantly for the application envisaged, a mapping function could also be pretrained on healthy participants and then tested in terms of its ability to predict brain responses in patients. Accurate brain response prediction would indicate that the patient's brain responses to language resemble those of the healthy participants, indicating conscious understanding. The use of a pretrained mapping function can be particularly useful when only limited amounts of data can be obtained from a given patient. In such cases, it may not be possible to fit a reliable mapping function using only data from the patients. A pretrained mapping function can therefore be useful in such situations.

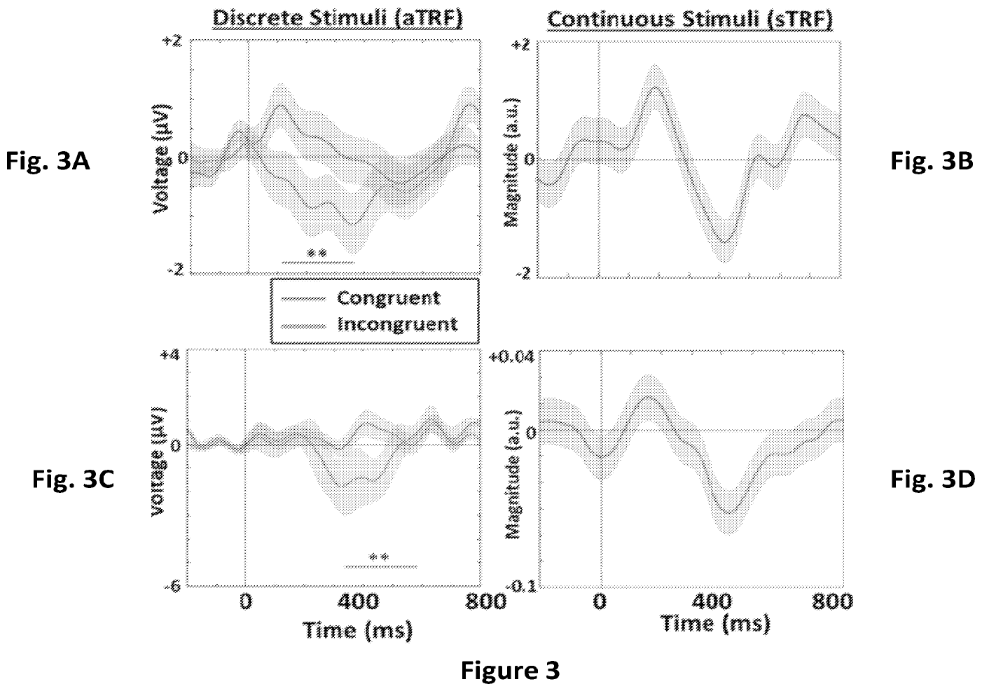

FIGS. 3A-3D illustrates plots of temporal response functions of two subjects, subjects 1 and 2 (S1 and S2) to discrete (FIGS. 3A and 3B) and continuous (FIGS. 3C and 3D) speech stimuli. The discrete stimuli presented to subjects S1 and S2 consisted of sets of sentences that ended with either congruous words or non-congruous words. The temporal response function demonstrated a significant negative spike for both subjects S1 and S2 in the range of 300-400 ms following a non-congruous stimulus, whereas the spike is absent or much less pronounced in response to congruous speech stimuli. In other subjects, the temporal response function can demonstrate a significant negative spike in a range lesser than 300 ms or greater than 400 ms. The TRF function was calculated using the regression analysis described in relation to FIG. 2 determining the relationship between neural response and a semantic metric associated with a spoken word relative to prior context. The peak in the TRF functions in response to non-congruous speech is interpreted as being indicative of the subjects' brains recognizing the semantic incongruity, implicating some level of semantic processing capability. FIGS. 3C and 3D represent the TRF signals of the same subjects S1 and S2 as a result of the subjects being exposed to natural language speech, with the TRF function also being calculated as described in relation to FIG. 2. The plots of the TRF functions in FIGS. 3C and 3D, like those resulting from evaluation of neural response to non-congruous speech, included a negative spike at about 400 ms delayed from exposure to content words (i.e., words having significant semantic meaning). Accordingly, it was determined that evaluating a TRF of a subject generated from a response to natural speech can effectively assess the subject's ability to semantically process speech.

Assessing semantic processing levels of subjects based on natural language speech, rather than discrete speech such as congruous and non-congruous sentences, can have several advantages. First, evaluations can be conducted continuously throughout the day as the subject is exposed to speech stimuli, provided the speech is recorded and transcribed, time-locked with recorded neural activity. This reduces the need to schedule evaluations. In addition, no special training is needed to be given to the speaker providing the stimulus and no special content need be generated or employed. The speaker can be the subject's regular caregiver, a family member, or even a recorded content item of interest, such as an audiobook, television program, or other recorded media item. Thus, the significant amounts of subject data can be accumulated and tracked over long periods of time to identify periods of increased processing capability, decreased processing capability and potential correlations of such changes to other external stimuli, environmental changes, or administration of treatments. In total, the use of natural speech to assess semantic understanding in subjects suffering form ABI or other diseases of consciousness provides a lower cost, more easily administered, and more informative assessment of the subjects brain functionality, thereby increasing the potential for appropriate assessment of the underlying physical impairment and/or mental health of the subject.

The systems and methods of the disclosure also provides assessment of comprehension levels in real time in brain injury and in non-brain injury settings, such as in developing children and in subjects with developmental disorders, such as autism, language or communication disorders, or other cognitive impairments or developmental impairments.

### Example Applications

The systems and methods disclosed herein may be applied to various applications without departing from the spirit of the disclosure. The forgoing applications and implementations are therefore to be considered in all respects illustrative, rather than limiting of the invention. In some implementations, the temporal response function assessment systems and methods described can be employed to provide EEG, MEG, and ECoG based diagnostics for assessing language processing in subjects. In some implementations, systems and methods according to the present disclosure provide clinical diagnostics of conditions such as disorders of consciousness following traumatic or ischemic brain injury, the operative monitoring of anesthesia, the evaluation of speech and cognitive function in patients with strokes and aphasia, the evaluation of novel or existing treatments and pharmacological drugs for neurological conditions, and a diagnostic to evaluate sports or military related brain-injury.

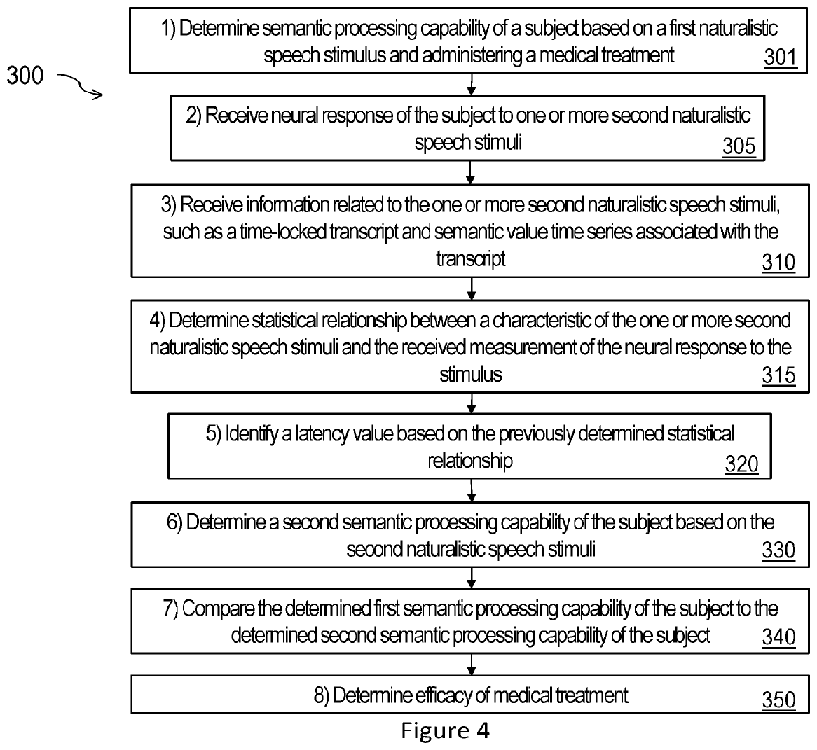

In some implementations, measuring the temporal response function of a patient natural speech stimuli may be utilized for the assessment of the efficacy of treatments for neurological conditions. Referring to FIG. 2, FIG. 4 shows a flow diagram of an example method 300 for determining the efficacy of a medical treatment based on a subject's determined semantic processing capability. First, method 300 includes determining a first semantic processing capability of a subject based on a first naturalistic speech stimulus and administering a medical treatment (step 301), for example using the methodology discussed in relation to FIG. 2 and the system of FIG. 1. Next, method 300 includes receiving a neural response of the subject to one or more second naturalistic speech stimuli (step 305). The method 300 also includes receiving information related to the one or more second naturalistic speech stimuli, such as a time-locked transcript and semantic value time series associated with the transcript (step 310). In a manner similar to that described FIG. 2, method 300 also includes determining a statistical relationship between a characteristic of the one or more second naturalistic speech stimuli (i.e., the semantic value of each word) and the received measurement of the neural response to the stimulus (step 315), resulting in a temporal response function. Similar to the method 200 in FIG. 2, method 300 also includes identifying a latency value based on the previously determined statistical relationship from step 315 (step 320), Next, method 300 includes determining a second semantic processing capability of the subject based on the second naturalistic speech stimuli (step 330). Next, method 300 includes comparing the determined first semantic processing capability of the subject from step 301 to the determined second semantic processing capability of the subject (step 340). Then, method 300 includes determining an efficacy of the medical treatment from step 301 based on the comparison from step 340 (step 350).

In some implementations, the use of the proposed systems and methods for providing a semantic understanding diagnostic for the assessment of cognitive function in brain injured patients may occur in an outpatient screening, neuro-intensive care unit (ICU), chronic care facilities, primary care settings, or sports and military centers. In some implementations, the system and methods of the current disclosure can track the cognitive function of patients in the operating room of hospitals. The sensory evoked response can be tracked via EEG during an operation requiring sedation by tracking the patient's cognitive function throughout the course of the sedation based on their response to the naturalistic speech stimuli. In some implementations, systems and methods according to the present disclosure can provide an EEG based diagnostic for intensive care unit monitoring. In the intensive care unit, the current system and methods can monitor and diagnose a severely brain-injured patient's semantic processing function and track their prognosis over time. For example, coma patients with a preserved semantic processing response to naturalistic speech may have an improved outcome compared to patients without a preserved semantic processing response to a naturalistic speech stimulus. Similarly, in some implementations, the systems and methods according to the present disclosure provide an EEG or ECOG based diagnostic of cognitive brain function for anesthetic depth and operative monitoring.

FIG. 5 illustrates a block diagram of an example computing system 1500. In some implementations, the computing system 1500 may be utilized in implementing the diagnostic methods in FIGS. 2 and 4.

In broad overview, the computing system 1510 includes at least one processor 1550 for performing actions in accordance with instructions and one or more memory devices 1570 or 1575 for storing instructions and data. The illustrated example computing system 1510 includes one or more processors 1550 in communication, via a bus 1515, with at least one network interface controller 1520 with network interface ports 1522(a-n) connecting to other computing devices 1524(a-n), memory 1570, and any other devices 1580, e.g., an I/O interface. Generally, a processor 1550 will execute instructions received from memory. The processor 1550 illustrated incorporates, or is directly connected to, cache memory 1575.

In more detail, the processor 1550 may be any logic circuitry that processes instructions, e.g., instructions fetched from the memory 1570 or cache 1575. In many embodiments, the processor 1550 is a microprocessor unit or special purpose processor. The computing device 1500 may be based on any processor, or set of processors, capable of operating as described herein. In some implementations, the processor 1550 can be capable of executing the diagnostic methods shown in FIG. 2 and FIG. 6. The processor 1550 may be a single core or multi-core processor. The processor 1550 may be multiple processors. In some implementations, the processor 1550 can be configured to run multi-threaded operations. In some implementations, the processor 1550 may host one or more virtual machines or containers, along with a hypervisor or container manager for managing the operation of the virtual machines or containers. In such implementations, one or more of the methods 200 and 300 shown in FIG. 2 and FIG. 4 can be implemented within the virtualized or containerized environments provided on the processor 1550.

The memory 1570 may be any device suitable for storing computer readable data. The memory 1570 may be a device with fixed storage or a device for reading removable storage media. Examples include all forms of non-volatile memory, media and memory devices, semiconductor memory devices (e.g., EPROM, EEPROM, SDRAM, and flash memory devices), magnetic disks, magneto optical disks, and optical discs (e.g., CD ROM, DVD-ROM, and BluRay® discs). A computing system 1500 may have any number of memory devices 1570. In some implementations, the memory 1570 supports virtualized or containerized memory accessible by virtual machine or container execution environments provided by the computing system 1510.

The cache memory 1575 is generally a form of computer memory placed in close proximity to the processor 1550 for fast read times. In some implementations, the cache memory 1575 is part of, or on the same chip as, the processor 1550. In some implementations, there are multiple levels of cache 1575, e.g., L2 and L3 cache layers.

The network interface controller 1520 manages data exchanges via the network interfaces 1522(a-n) (also referred to as network interface ports). The network interface controller 1520 handles the physical and data link layers of the OSI model for network communication. In some implementations, some of the network interface controller's tasks are handled by the processor 1550. In some implementations, the network interface controller 1520 is part of the processor 1550. In some implementations, a computing system 1510 has multiple network interface controllers 1520. The network interfaces 1522(a-n) are connection points for physical network links. In some implementations, the network interface controller 1520 supports wireless network connections and an interface port 1522 is a wireless receiver/transmitter. Generally, a computing device 1510 exchanges data with other computing devices 1512(a-n) via physical or wireless links to a network interfaces 1522(a-n). In some implementations, the network interface controller 1520 implements a network protocol such as Ethernet.

The other computing devices 1524(a-n) are connected to the computing device 1510 via a network interface port 1522. The other computing devices 1524(a-n) may be peer computing devices, network devices, or any other computing device with network functionality. For example, a first computing device 1524(a) may be a network device such as a hub, a bridge, a switch, or a router, connecting the computing device 1510 to a data network such as the Internet.

The other devices 1580 may include an I/O interface, external serial device ports, and any additional co-processors. For example, a computing system 1510 may include an interface (e.g., a universal serial bus (USB) interface) for connecting input devices (e.g., a keyboard, microphone, mouse, or other pointing device), output devices (e.g., video display, speaker, or printer), or additional memory devices (e.g., portable flash drive or external media drive). In some implementations, a computing device 1500 includes an additional device 1580 such as a coprocessor, e.g., a math co-processor can assist the processor 1550 with high precision or complex calculations.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted that the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A method comprising:
recording neural data of a subject while the subject is presented with a natural speech stimulus;
obtaining the transcript of the natural speech stimulus, with data indicating onset time of words included in transcript;
calculating using a processor a semantic metric for the words used in the transcript based on the meaning of the words;
creating using a processor a time series of impulses based on the onset times of the words in the transcript and the semantic metrics calculated for the words;
calculating by the processor a stimulus-response mapping function by regressing the recorded neural data onto the time series; and
determining by the processor the semantic processing capability of the subject based on the calculated mapping function.

2. The method of claim 1, wherein the stimulus-response mapping function is calculated using regularized linear regression.

3. The method of claim 1, wherein the semantic processing capability of the subject is determined based on the identification or absence of a peak in the stimulus-response mapping function at a time of about 300 to about 400 ms.

4. The method of claim 1, wherein determining the semantic processing capability of the subject based on the calculated mapping function comprises determining the mapping function having a statistically significant correlation between the neural data and the time series than a random mapping function.

5. The method of claim 1, wherein the semantic metric for a word in the transcript is based on the probability that a given word will follow preceding words in the transcript.

6. The method of claim 1, wherein the semantic metric for a word in the transcript is based on a difference between a vector indicative of the semantic meaning of the word relative to one or more semantic vectors or combinations thereof corresponding to preceding words in the transcript.

7. The method of claim 1, further comprising:
after determining the semantic processing capability of the subject, administering a medical treatment to the subject;
after administering the treatment, receiving, by the processor, a measurement of a second neural response of a subject to one or more second naturalistic speech stimuli;
receiving, by the processor, information related to the one or more second naturalistic speech stimuli;
determining, by the processor, a second statistical relationship between semantic contribution of words in the second naturalistic speech stimuli to the second neural response;
identifying, by the processor, a second semantic processing capability of the subject based on the determined second statistical relationship; and
comparing, by the processor, the determined first semantic processing capability to the determined second semantic processing capability function;
determining, by the processor, an efficacy of the medical treatment based on the comparison; and,
outputting, by the processor, the determined efficacy of the medical treatment.

8. The method of claim 1, wherein calculating the semantic metric for the words used in the transcript based on their meaning comprises calculating a difference between a measure associated with the meaning of each word used in the transcript and a preceding context.

9. A system comprising:
a processor for generating a transcript of natural speech stimulus presented to a subject and for annotating the transcript with an onset time of words in the transcript;
a neural sensor for recording a neural response of the subject to the natural speech stimulus;
one or more processors implementing a processing unit configured to determine an indication of the semantic processing capability of a subject to the one or more naturalistic sensory stimuli by:
receiving a measurement of a neural response in the subject exposed to the one or more naturalistic speech stimuli output by the neural sensor;
determining a statistical relationship between the semantic contribution of words in the transcript to the naturalistic sensory stimuli and the measurement of the neural response of the subject;
determining an indication of the semantic processing capability of the subject based on the statistical relationship; and,
an output module for outputting the determined semantic processing capability.

10. The system of claim 1, wherein the semantic processing capability of the subject is determined based on the identification or absence of a peak in the stimulus-response mapping function at a time of about 300 to about 400 ms.

11. The system of claim 1, wherein determining the semantic processing capability of the subject based on the calculated mapping function comprises determining the mapping function having a statistically significant correlation between the neural data and the time series than a random mapping function.

12. The system of claim 1, further comprising:
after determining the semantic processing capability of the subject, administering a medical treatment to the subject;
after administering the treatment, receiving, by the processor, a measurement of a second neural response of a subject to one or more second naturalistic speech stimuli;
receiving, by the processor, information related to the one or more second naturalistic speech stimuli;
determining, by the processor, a second statistical relationship between semantic contribution of words in the second naturalistic speech stimuli to the second neural response;
identifying, by the processor, a second semantic processing capability of the subject based on the determined second statistical relationship; and
comparing, by the processor, the determined first semantic processing capability to the determined second semantic processing capability function;
determining, by the processor, an efficacy of the medical treatment based on the comparison; and,
outputting, by the processor, the determined efficacy of the medical treatment.

13. A non-transitory computer-readable medium storing code, the code comprising instructions executable to:
record neural data of a subject while the subject is presented with a natural speech stimulus;
obtain the transcript of the natural speech stimulus, with data indicating onset time of words included in transcript;
calculate, using a processor, a semantic metric for the words used in the transcript by relating the meaning of the words;
create, using a processor, a time series of impulses based on the onset times of the words in the transcript and the semantic metrics calculated for the words;
calculate, by the processor, a stimulus-response mapping function by regressing the recorded neural data onto the time series; and
determine, by the processor, the semantic processing capability of the subject based on the calculated mapping function.

14. The non-transitory computer-readable medium of claim 1, wherein the stimulus-response mapping function is calculated using regularized linear regression.

15. The non-transitory computer-readable medium of claim 1, wherein the semantic processing capability of the subject is determined based on the identification or absence of a peak in the stimulus-response mapping function at a time of about 300 to about 400 ms.

16. The non-transitory computer-readable medium of claim 1, wherein the semantic processing capability of the subject is determined based on the calculated mapping function having a statistically significant correlation between the neural data and the time series than a random mapping function.

17. The non-transitory computer-readable medium of claim 1, wherein the semantic metric for a word in the transcript is based on the probability that a given word will follow preceding words in the transcript.

18. The non-transitory computer-readable medium of claim 1, wherein the semantic metric for a word in the transcript is based on a difference between a vector indicative of the semantic meaning of the word relative to one or more semantic vectors or combinations thereof corresponding to preceding words in the transcript.

19. The non-transitory computer-readable medium of claim 1, the code further comprising instructions executable to:
administer a medical treatment to the subject after determining the semantic processing capability of the subject;
receive, by the processor, a measurement of a second neural response of a subject to one or more second naturalistic speech stimuli after administering the treatment;
receive, by the processor, information related to the one or more second naturalistic speech stimuli;
determine, by the processor, a second statistical relationship between semantic contribution of words in the second naturalistic speech stimuli to the second neural response;
identify, by the processor, a second semantic processing capability of the subject based on the determined second statistical relationship; and
compare, by the processor, the determined first semantic processing capability to the determined second semantic processing capability function;
determine, by the processor, an efficacy of the medical treatment based on the comparison; and,
output, by the processor, the determined efficacy of the medical treatment.

20. The non-transitory computer-readable medium of claim 1, wherein relating the meaning of the words used in the transcript is based on a difference between the meaning of the words used in the transcript and a preceding context of the words used in the transcript.
